(19) United States
(12) Patent Application Publication
(10) Pub. No.: US 2022/0031636 A1
(43) Pub. Date: Feb. 3, 2022

(54) COMPOUNDS THAT ENHANCE THE ACTION OF METFORMIN

(71) Applicant: SOCIETE DES PRODUiTS NESTLE S.A., Vevey (CH)

(72) Inventors: Denis Marcel Barron, Lutry (CH); Nicolas Bonhoure, Chavornay (CH); Yann Ratinaud, Morges (CH); Kei Sakamoto, Kobenbavn O (DK); Matthew Sanders, Epalinges (CH)

(21) Appl. No.: 17/280,319

(22) PCT Filed: Sep. 25, 2019
(86) PCT No.: PCT/EP2019/075904

§ 371 Date: Mar. 26, 2021

(30) Foreign Application Priority Data
Sep. 28, 2018 (EP) 18197748.9

(51) Int. Cl. A61K 31/085; A61K 31/155

(52) U.S. Cl. CPC A61K 31/085; A61K 31/155

(57) ABSTRACT

The invention relates in general to a medicament comprising Metformin or a salt thereof; and a compound having the general formula (I) or a salt thereof. The invention further relates to a pharmaceutical composition comprising the medicament for use in the prevention or treatment of a metabolic disease or a cardiovascular disease.

### INTRODUCTION

According to international guidelines, metformin, a biguanide, is the recommended first-line oral therapy for the treatment of type 2 diabetes (PMID: 18941734) mainly due to its efficacy, very low risk of hypoglycemia, and low cost. In addition, there are likely to be other beneficial effects, including a reduction in cardiovascular disease and cancer incidence (PMID: 15849206).

Although rare, metformin has some risks, including life-threatening lactic acidosis and other unwanted effects. Gastrointestinal (GI) side effects are the main complaint of patients and the most common GI side effects are diarrhea, nausea, and vomiting (with a prevalence of 2%-63% according to recent literature) which occur more than with all other oral antidiabetic agents and impair compliance. As a result, approximately 5% of patients discontinue therapy. Alternative solutions, either to mimic or enhance the effect of metformin at lower dose with other drugs/interventions would therefore be desirable.

The molecular mechanism by which metformin elicits its therapeutic effect is still not fully understood. One of the most established cellular actions of metformin is to inhibit de novo lipogenesis and thus reduce excess lipids in the liver leading to improvement of insulin sensitivity through activation of AMP-activated protein kinase (AMPK) (PMID: 11602624, 24185692). AMPK is a master regulator of cellular energy homeostasis and is considered a key drug target of metabolic disorders (i.e. obesity, type 2 diabetes, cardiovascular disease). Currently, there is no direct AMPK-activating drug available to treat metabolic disorders despite intensive efforts continuously made by the pharmaceutical industry.

The present invention provides a solution, enabling a lower metformin dose by combining treatment with direct natural AMPK activators.

### SUMMARY OF INVENTION

Medicament

The present invention relates in general to a medicament comprising (i) an agent suitable for the prevention or treatment of a metabolic disease or a cardiovascular disease, and (ii) a compound having the general formula (I) as described herein.

In one embodiment, compound (i) is an anti-diabetic agent. In one embodiment, compound (i) is a biguanide compound, for example Metformin or a salt thereof.

In particular, the present invention relates to a medicament comprising

(i) Metformin, or a salt thereof; and

(ii) a compound having a general formula (I)

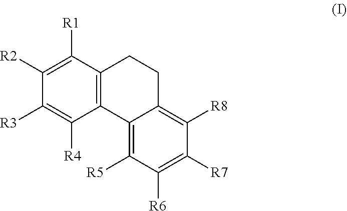

or a salt thereof,

wherein, in compound (ii), R1, R2, R3, R4, R5, R6, R7, and R8 are each independently H; OH; OMe; O-glycoside; a halogen; an aldehyde; a carboxylic acid; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched C2 to C20 alkenyl; an optionally substituted and/or optionally branched C4 to C20 polyalkenyl; an optionally substituted and/or optionally branched C2 to C20 alkynyl; or an optionally substituted and/or optionally branched C4 to C20 polyalkynyl.

In some embodiments, R1, R2, R3, R4, R7, and R8 are each independently H; OH; OMe; O-glycoside; a halogen; an aldehyde; a carboxylic acid; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched C2 to C20 alkenyl; an optionally substituted and/or optionally branched C4 to C20 polyalkenyl; an optionally substituted and/or optionally branched C2 to C20 alkynyl; or an optionally substituted and/or optionally branched C4 to C20 polyalkynyl; R5 is H; OMe; O-glycoside; a halogen; an aldehyde; a carboxylic acid; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched C2 to C20 alkenyl; an optionally substituted and/or optionally branched C4 to C20 polyalkenyl; an optionally substituted and/or optionally branched C2 to C20 alkynyl; or an optionally substituted and/or optionally branched C4 to C20 polyalkynyl; R6 is H; OH; O-glycoside; a halogen; an aldehyde; a carboxylic acid; a primary, secondary, or tertiary amine; a primary or secondary amide; a cyano; a nitro; a sulfonate; a sulfate; an optionally substituted and/or optionally branched C1 to C20 alkyl; an optionally substituted and/or optionally branched C2 to C20 alkenyl; an optionally substituted and/or optionally branched C4 to C20 polyalkenyl; an optionally substituted and/or optionally branched C2 to C20 alkynyl; or an optionally substituted and/or optionally branched C4 to C20 polyalkynyl in compound (ii).

In some embodiments, R1, R2, R3, R4, R7, and R8 are each independently H; OH; OMe; O-glycoside; a halogen; an aldehyde; a carboxylic acid; a primary, secondary, or tertiary amine; a primary or secondary amide; a cyano; a nitro; a sulfonate; a sulfate; R5 is H; OMe; O-glycoside; a halogen; an aldehyde; a carboxylic acid; a primary, secondary, or tertiary amine; a primary or secondary amide; a cyano; a nitro; a sulfonate; a sulfate; R6 is H; OH; O-glycoside; a halogen; an aldehyde; a carboxylic acid; a primary, secondary, or tertiary amine; a primary or secondary amide; a cyano; a nitro; a sulfonate; a sulfate in compound (ii).

In some embodiments, R1, R2, R3, and R8 are each independently H; OH; OMe; O-glycoside; a halogen; an aldehyde; a carboxylic acid; a primary, secondary, or tertiary amine; a primary or secondary amide; a cyano; a nitro; a sulfonate; a sulfate; R4 is H; OH; O-glycoside; a halogen; an aldehyde; a carboxylic acid; a primary, secondary, or tertiary amine; a primary or secondary amide; a cyano; a nitro; a sulfonate; a sulfate; R5 is H; OMe; O-glycoside; a halogen; an aldehyde; a carboxylic acid; a primary, secondary, or tertiary amine; a primary or secondary amide; a cyano; a nitro; a sulfonate; a sulfate; R6 is H; OH; O-glycoside; a halogen; an aldehyde; a carboxylic acid; a primary, secondary, or tertiary amine; a primary or secondary amide; a cyano; a nitro; a sulfonate; a sulfate; and R7 is H; OH; O-glycoside; a halogen; an aldehyde; a carboxylic acid; a primary, secondary, or tertiary amine; a primary or secondary amide; a cyano; a nitro; a sulfonate; a sulfate; in compound (ii).

In some embodiments, R1, R3, and R8 are each independently H; OH; OMe; O-glycoside; a halogen; an aldehyde; a carboxylic acid; a primary, secondary, or tertiary amine; a primary or secondary amide; a cyano; a nitro; a sulfonate; a sulfate; R2 is OH; OMe; O-glycoside; a halogen; an aldehyde; a carboxylic acid; a primary, secondary, or tertiary amine; a primary or secondary amide; a cyano; a nitro; a sulfonate; a sulfate; R4 is H; OH; O-glycoside; a halogen; an aldehyde; a carboxylic acid; a primary, secondary, or tertiary amine; a primary or secondary amide; a cyano; a nitro; a sulfonate; a sulfate; R5 is H; OMe; O-glycoside; a halogen; an aldehyde; a carboxylic acid; a primary, secondary, or tertiary amine; a primary or secondary amide; a cyano; a nitro; a sulfonate; a sulfate; R6 is H; OH; O-glycoside; a halogen; an aldehyde; a carboxylic acid; a primary, secondary, or tertiary amine; a primary or secondary amide; a cyano; a nitro; a sulfonate; a sulfate; and R7 is H; OH; O-glycoside; a halogen; an aldehyde; a carboxylic acid; a primary, secondary, or tertiary amine; a primary or secondary amide; a cyano; a nitro; a sulfonate; a sulfate; in compound (ii).

In some embodiments, R1, R3, and R8 are each independently H; OH; OMe; O-glycoside; a halogen; an aldehyde; a carboxylic acid; R2 is OH; OMe; O-glycoside; a halogen; an aldehyde; a carboxylic acid; R4 is H; OH; O-glycoside; a halogen; an aldehyde; a carboxylic acid; R5 is H; OMe; O-glycoside; a halogen; an aldehyde; a carboxylic acid; R6 is H; OH; O-glycoside; a halogen; an aldehyde; a carboxylic acid; and R7 is H; OH; O-glycoside; a halogen; an aldehyde; a carboxylic acid in compound (ii).

In some embodiments, R1, R3, and R8 are each independently H; OH; OMe; O-glycoside; an aldehyde; a carboxylic acid; R2 is OH; OMe; O-glycoside; an aldehyde; a carboxylic acid; R4 is H; OH; O-glycoside; an aldehyde; a carboxylic acid; R5 is H; OMe; O-glycoside; an aldehyde; a carboxylic acid; R6 is H; OH; O-glycoside; an aldehyde; a carboxylic acid; and R7 is H; OH; O-glycoside; an aldehyde; a carboxylic acid in compound (ii).

In some embodiments, R1, R3, and R8 are each independently H; OH; OMe; R2 is OH; OMe; R4 is H; OH; R5 is H; OMe; R6 is H; OH; and R7 is H; OH in compound (ii).

In some embodiments, R1, R3, R6 and R8 are each independently H; R2 is OH; OMe; R4 is OH; R5 is H; OMe; and R7 is H; OH in compound (ii).

In one embodiment, compound (ii) is Lusianthridin (7-Methoxy-9,10-dihydrophenanthrene-2,5-diol, CAS number 87530-30-1) and has the formula

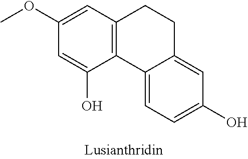 [SMILES: COc1cc(O)c2c(c1)CCc1cc(O)ccc1-2]

In one embodiment, compound (i) and compound (ii) of the medicament are combined in a single preparation. In one embodiment, compound (i) and compound (ii) of the medicament are combined in a single preparation with at least one other anti-diabetic agent different to compound (i).

In one embodiment, compound (i) and compound (ii) of the medicament are separately formulated into compositions and used in combination. In one embodiment, compound (i) and compound (ii) of the medicament are separately formulated into compositions and used in combination with at least one other anti-diabetic agent different to compound (i).

In some embodiments, the at least one other anti-diabetic agent is a sulfonylurea compound, or a salt thereof. In some embodiments, the at least one other anti-diabetic agent is a thiazolidinedione compound, or a salt thereof. In some embodiments, the at least one other anti-diabetic agent is a alpha-glucosidase inhibitor compound, or a salt thereof. In some embodiments, the at least one other anti-diabetic agent is a GLP-1 analog compound, or a salt thereof.

Pharmaceutical Composition

There is also provided a pharmaceutical composition comprising a medicament of the invention as described herein, in a pharmaceutically acceptable form.

In some embodiments, the pharmaceutical composition comprises (i) an agent suitable for the prevention or treatment of a metabolic disease or a cardiovascular disease or a pharmaceutically acceptable salt thereof, and (ii) a compound having the general formula (I) as described herein or a pharmaceutically acceptable salt thereof.

In some embodiments, compound (i) is an anti-diabetic agent. In some embodiments, the anti-diabetic agent is a biguanide or a pharmaceutically acceptable salt thereof. In some embodiments, the biguanide is Metformin or a pharmaceutically acceptable salt thereof.

In some embodiments, in compound (ii), R1, R3, and R8 are each independently H; OH; OMe; O-glycoside; a halogen; an aldehyde; a carboxylic acid; R2 is OH; OMe; O-glycoside; a halogen; an aldehyde; a carboxylic acid; R4 is H; OH; O-glycoside; a halogen; an aldehyde; a carboxylic acid; R5 is H; OMe; O-glycoside; a halogen; an aldehyde; a carboxylic acid; R6 is H; OH; O-glycoside; a halogen; an aldehyde; a carboxylic acid; and R7 is H; OH; O-glycoside; a halogen; an aldehyde; a carboxylic acid.

In some embodiments, R1, R3, and R8 are each independently H; OH; OMe; O-glycoside; an aldehyde; a carboxylic acid; R2 is OH; OMe; O-glycoside; an aldehyde; a carboxylic acid; R4 is H; OH; O-glycoside; an aldehyde; a carboxylic acid; R5 is H; OMe; O-glycoside; an aldehyde; a carboxylic acid; R6 is H; OH; O-glycoside; an aldehyde; a carboxylic acid; and R7 is H; OH; O-glycoside; an aldehyde; a carboxylic acid.

In some embodiments, R1, R3, and R8 are each independently H; OH; OMe; R2 is OH; OMe; R4 is H; OH; R5 is H; OMe; R6 is H; OH; and R7 is H; OH.

In some embodiments, R1, R3, R6 and R8 are each independently H; R2 is OH; OMe; R4 is OH; R5 is H; OMe; and R7 is H; OH.

In some embodiments, the pharmaceutical composition comprises (i) Metformin or a pharmaceutically acceptable salt thereof, and (ii) Lusianthridin or a pharmaceutically acceptable salt thereof.

In one embodiment, compound (i) and compound (ii) of the pharmaceutical composition are combined in a single preparation. In one embodiment, compound (i) and compound (ii) of the pharmaceutical composition are combined in a single preparation with at least one other anti-diabetic agent different to compound (i).

In one embodiment, compound (i) and compound (ii) of the pharmaceutical composition are separately formulated into compositions and used in combination. In one embodiment, compound (i) and compound (ii) of the pharmaceutical composition are separately formulated into compositions and used in combination with at least one other anti-diabetic agent different to compound (i).

In some embodiments, the at least one other anti-diabetic compound is a sulfonylurea compound, or a pharmaceutically salt thereof. In some embodiments, the at least one other anti-diabetic agent is a thiazolidinedione compound, or a pharmaceutically acceptable salt thereof. In some embodiments, the at least one other anti-diabetic agent is a alpha-glucosidase inhibitor compound, or a pharmaceutically acceptable salt thereof. In some embodiments, the at least one other anti-diabetic agent is a GLP-1 analog compound, or a pharmaceutically acceptable salt thereof.

There is also provided a pharmaceutical composition as described herein for use in the prevention or treatment of a metabolic disease or a cardiovascular disease.

In one embodiment, the metabolic disease is diabetes. In one embodiment, the metabolic disease is nonalcoholic fatty liver disease (NAFLD). In one embodiment, the metabolic disease is nonalcoholic steatohepatitis (NASH).

In one embodiment, the pharmaceutical composition is for use in the prevention or treatment of diabetes or NAFLD by increasing the inhibition of lipogenesis.

In one embodiment, the pharmaceutical composition is for use in the prevention or treatment of diabetes or NAFLD by increasing the inhibition of lipogenesis in liver cells.

In one embodiment, the pharmaceutical composition is for use in the prevention or treatment of diabetes by (i) increasing the inhibition of lipogenesis in liver cells and (ii) improvement of insulin sensitivity by activation of AMP-activated protein kinase (AMPK).

In one embodiment, compound (i) can be used at a sub-pharmacological dose for substantially the same degree of inhibition of lipogenesis when used in combination with compound (ii), compared to that of a pharmaceutical composition comprising a pharmacological dose of compound (i) but wherein compound (ii) is absent.

In one embodiment, the pharmaceutical composition is for use according to the invention, wherein the subject is a human.

In some embodiments, the pharmaceutical composition is for use according to the invention, wherein the subject has or is at risk of developing a further condition, disorder, or disease, wherein said further condition, disorder or disease relates to cardio-metabolic health, cancer, obesity, non-alcoholic fatty liver disease, and/or cardiovascular disease.

In some embodiments, the pharmaceutical composition is for use according to the invention wherein the further condition, disorder, or disease relates to cardio-metabolic health, and/or cancer.

In one embodiment, the pharmaceutical composition is for use according to the invention, wherein compound (i) is used at a sub-pharmacological dose.

In one embodiment, the pharmaceutical composition is for use according to the invention, wherein metformin is used at a sub-pharmacological dose, said sub-pharmacological dose being not less than about 50% of the standard pharmacological dose for the subject.

In some embodiments, the pharmaceutical composition is for use according to the invention, wherein the sub-pharmacological dose of metformin reduces the incidence of side effects associated with metformin use, and wherein the side effects are lactic acidosis, and/or gastro-intestinal side effects, particularly diarrhea, nausea, and vomiting.

In some embodiments, the pharmaceutical composition for use according to the invention is Glucophage or Glucophage XR. A typical standard pharmacological dose of Glucophage is 1 g twice per day. A typical standard pharmacological dose of Glucophage XR is maximum 2 g, once per day with an evening meal.

In one embodiment, compound (i) and compound (ii) are combined in a single preparation. In one embodiment, compound (i) and compound (ii) are combined in a single preparation with at least one other anti-diabetic agent different to compound (i).

In one embodiment, compound (i) and compound (ii) are separately formulated into compositions and used in combination. In one embodiment, compound (i) and compound (ii) are separately formulated into compositions and used in combination with at least one other anti-diabetic agent different to compound (i).

In some embodiments, the at least one other anti-diabetic agent is a sulfonylurea compound, or a pharmaceutically acceptable salt thereof. In some embodiments, the at least one other anti-diabetic agent is a thiazolidinedione compound, or a pharmaceutically acceptable salt thereof. In some embodiments, the at least one other anti-diabetic agent is a alpha-glucosidase inhibitor compound, or a pharmaceutically acceptable salt thereof. In some embodiments, the at least one other anti-diabetic agent is a GLP-1 analog compound, or a pharmaceutically acceptable salt thereof

Method of Preventing or Treating a Disease

There is also provided a method of preventing or treating a metabolic disease or a cardiovascular disease comprising the administration of a medicament or a pharmaceutical composition of the invention to a subject in need thereof, wherein the medicament or pharmaceutical composition comprises (i) an agent suitable for the prevention or treatment of a metabolic disease or a cardiovascular disease, and/or (ii) a compound having the general formula (I) as described herein.

In one embodiment, the metabolic disease is diabetes. In one embodiment, the method increases the inhibition of lipogenesis, for example in liver cells. In one embodiment, the method improves insulin sensitivity by activation of AMP-activated protein kinase (AMPK).

In one embodiment, compound (i) can be administered at a sub-pharmacological dose for substantially the same degree of inhibition of lipogenesis when used in combination with compound (ii), compared to that of a pharmaceutical composition comprising a pharmacological dose of compound (i) but wherein compound (ii) is absent.

In some embodiments, the subject has or is at risk of developing a further condition, disorder, or disease, wherein said further condition, disorder or disease relates to cardio-metabolic health, cancer, obesity, non-alcoholic fatty liver disease, and/or cardiovascular disease. In some embodiments, the further condition, disorder, or disease relates to cardio-metabolic health, and/or cancer.

In one embodiment, compound (i) is administered at a sub-pharmacological dose.

In one embodiment, compound (i) is metformin, wherein metformin is administered at a sub-pharmacological dose, said sub-pharmacological dose being not more than about 50% of the standard pharmacological dose for the subject.

In some embodiments, the sub-pharmacological dose of metformin reduces the incidence of side effects associated with metformin administration, and wherein the side effects are lactic acidosis, and/or gastro-intestinal side effects, particularly diarrhea, nausea, and vomiting.

The anti-diabetic agent Metformin as disclosed herein can be marketed in the form of its hydrochloride salt (Glucophage®), Bristol-Myers Squibb Company). In some embodiments, the pharmaceutical composition for use according to the invention is Glucophage or Glucophage XR. A typical standard pharmacological dose of Glucophage is 1 g twice per day. A typical standard pharmacological dose of Glucophage XR is maximum 2 g, once per day with an evening meal.

In one embodiment, compound (i) and compound (ii) are combined in a single preparation. In one embodiment, compound (i) and compound (ii) are combined in a single preparation with at least one other anti-diabetic agent different to compound (i).

In one embodiment, compound (i) and compound (ii) are separately formulated into compositions and administered in combination. In one embodiment, compound (i) and compound (ii) are separately formulated into compositions and administered in combination with at least one other anti-diabetic agent different to compound (i).

In some embodiments, the at least one other anti-diabetic compound is a sulfonylurea compound, or a pharmaceutically acceptable salt thereof. In some embodiments, the at least one other anti-diabetic agent is a thiazolidinedione compound, or a pharmaceutically acceptable salt thereof. In some embodiments, the at least one other anti-diabetic agent is a alpha-glucosidase inhibitor compound, or a pharmaceutically acceptable salt thereof. In some embodiments, the at least one other anti-diabetic agent is a GLP-1 analog compound, or a pharmaceutically acceptable salt thereof.

There is also provided a method for first line treatment of a metabolic disease in a subject, which comprises administering a drug to a subject in need of treatment, as first line therapy, a therapeutically effective dose of a pharmaceutical formulation comprising a sub-pharmacological dose of metformin and a compound of general formula (I) as described herein, which is an AMPK activator.

In one embodiment, the metabolic disease is diabetes. In one embodiment, the human subject is drug naive.

### DETAILED DESCRIPTION

The term “alkyl” refers to a branched or unbranched saturated hydrocarbon chain having from 1 to 20 carbon atoms, or from 1 to 15 carbon atoms, or from 1 to 10 carbon atoms, or from 1 to 8 carbon atoms, or from 1 to 6 carbon atoms, or from 1 to 4 carbon atoms. This term is exemplified by groups such as methyl, ethyl, n-propyl, iso-propyl, n-butyl, iso-butyl, t-butyl, n-hexyl, n-decyl, tetradecyl, and the like.

The term “substituted alkyl” refers to:

1) an alkyl chain as defined above, having 1, 2, 3, 4 or 5 substituents, (in some embodiments, 1, 2 or 3 substituents) selected from the group consisting of alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)— cycloalkyl, —S(O)-heterocyclyl, —S(O)-aryl, —S(O)-heteroaryl, —S(O)2-alkyl, —S(O)2-cycloalkyl, —S(O)2-heterocyclyl, —S(O)2-aryl and —S(O)2-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2; or

2) an alkyl chain as defined above that is interrupted by 1-10 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF3, amino, substituted amino, cyano, cycloalkyl, heterocyclyl, aryl, heteroaryl, and —S(O)n R<a>, in which R<a> is alkyl, aryl or heteroaryl and n is 0, 1 or 2; or

3) an alkyl chain as defined above that has both 1, 2, 3, 4 or 5 substituents as defined above and is also interrupted by 1-10 atoms (e.g. 1, 2, 3, 4 or 5 atoms) as defined above.

The term “alkenyl” refers to a type of alkyl chain in which two atoms of the alkyl group form a double bond that is not part of an aromatic group. That is, an alkenyl chain contains the pattern R—C(R)═C(R)—R, wherein R refers to the remaining portions of the alkenyl group, which may be the same or different. Non-limiting examples of an alkenyl chain include —CH═CH2, —C(CH3)═CH2, —CH═CHCH3, —C(CH3)=CHCH3, —CH2-CH═C(CH3)2, and —C(CH3)2—CH═CH2. The alkenyl moiety may be branched, straight chain, or cyclic (in which case, it would also be known as a “cycloalkenyl” group). Alkenyl groups can be optionally substituted.

The term “alkynyl” refers to a type of alkyl chain in which two atoms of the alkyl group form a triple bond. That is, an alkynyl group contains the pattern R—CC—R, wherein R refers to the remaining portions of the alkynyl group, which may be the same or different. Non-limiting examples of an alkynyl group include —C≡CH, —C≡CCH3 and —C≡CCH2CH3. The “R” portion of the alkynyl moiety may be branched, straight chain, or cyclic. Alkynyl groups can be optionally substituted.

The term “polyunsaturated” refers to

1) A chain known as polyalkenyl in which more than one pair of atoms of the alkyl group form a double bond that is not part of an aromatic group. That is, a polyalkenyl chain contains several R—C(R)═C(R)—R patterns, wherein R refers to the remaining portions of the alkenyl group, which may be the same or different. Non-limiting examples of a polyalkenyl chain include —CH═CH—CH═CH—, —CH2-CH═CCH3-CH2-CH2-CH═C(CH3)₂, and —CH2-CH═CCH3-CH2-CH2-CH═CCH3-CH2-CH2-CH═C(CH3)2. The polyalkenyl moiety may be branched, or straight chain. The polyalkenyl moiety containing two double bond may be cyclic (in which case, it would also be known as a “cyclodialkenyl” group). Non limiting example of cyclodialkenyl groups include cyclopentadiene and cyclohexadiene groups. Polyalkenyl groups can be optionally substituted.

2) A chain known as polyalkynyl in which more than one pair of atoms of the alkyl group form a triple bond. That is, a polyalkynyl group contains several patterns R—C≡C—R, wherein R refers to the remaining portions of the alkynyl group, which may be the same or different. Non-limiting example of a polyalkynyl group include —CH2-CH2-C≡C—C≡CH. The “R” portion of the polyalkynyl moiety may be branched, straight chain, or cyclic. Alkynyl groups can be optionally substituted.

3) A type of alkyl chain in which at least one pair of atoms of the alkyl group form a double bond and one pair of atoms of the alkyl group form a triple bond. That is, a polyunsaturated chain contains both R—C(R)═C(R)—R and R—C≡C—R patterns, wherein R refers to the remaining portions of the polyunsaturated chain, which may be the same or different. Non-limiting examples this type of polyunsaturated chain include —CH2-CH═CH—C≡CH. The “R” portion of the polyunsaturated moiety may be branched, straight chain, or cyclic. Polyunsaturated chains can be optionally substituted.

4) A polyunsaturated chain as defined above in paragraphs 1-3, that is interrupted by 1-10 atoms (e.g. 1, 2, 3, 4 or 5 atoms) independently chosen from oxygen, sulfur and NR<a>, where R<a> is chosen from hydrogen, alkyl, cycloalkyl, alkenyl, cycloalkenyl, alkynyl, aryl, heteroaryl and heterocyclyl.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can form part of a ring system. As used herein, the term “ring system” refers to two or more rings, wherein two or more of the rings are fused. The term “fused” refers to structures in which two or more rings share one or more bonds.

The term “halogen atom” may refer to a fluorine atom, a chlorine atom, a bromine atom or an iodine atom.

The term “analogue” as used herein is understood to refer to a compound having a structure similar to that of another one, but differing from it in respect of a certain component. A “derivative” is a compound that arises or is actually synthesized from a parent compound by replacement of one or more atoms with another atom or group of atoms.

The components of the chemical structures described herein can be further defined as follows: the term “Unsaturated” means it contains at least one, maximum eight double bond between carbon atoms. “Dehydration” means a loss of water between two neighbouring carbons, one bearing a hydroxyl and the other bearing at least one hydrogen, leading to the formation of a double bond. “Reduction” means addition of hydrogen to a double bond, leading to the formation of a single bond, typically reduction of a carbonyl to an alcohol, or an unsaturated chain to a saturated one. Carbon oxidation may be, for example, stepwise from a methyl to an alcohol, to an aldehyde, and finally to a carboxylic acid.

As used herein, the term “aldehyde” denotes an organic compound having the general structure —C—[C(═O)]n —H or H—[C(═O)]n-H (n is 1 or more and wherein the carbon atom bonded to the —[C(═O)]-n group is not double bonded to oxygen, sulfur, selenium, or tellurium, or triple bonded to nitrogen.

As used herein, the term “amine” denotes an organic compound having a nitrogen atom single or double bonded to a carbon atom and wherein the carbon atom bonded to the nitrogen atom is devoid of a double bond to oxygen, sulfur, selenium, or tellurium or triple bonded to nitrogen. In addition, those compounds wherein the same nitrogen atom is bonded to a —C(═X)— group (X is O, S, Se, or Te) and to a carbon atom which is not double bonded to oxygen, sulfur, selenium, or tellurium, are not considered as being amines, e.g., —C—NH—C(X═)—

As used herein, the term “cyano” denotes a triple bond between an adjacent carbon and nitrogen atom.

As used herein, the term “carboxylic acid” denotes the presence of a —C(═O)OH group.

As used herein, the term “metformin” refers to metformin or a salt thereof such as the hydrochloride salt, the metformin (2:1) fumarate salt, and the metformin (2:1) succinate salt, the hydrobromide salt, the p-chlorophenoxy acetate or the embonate, and other known metformin salts of mono and dibasic carboxylic acids, all of which salts are collectively referred to as metformin. Metformin as referred to herein may be the metformin hydrochloride salt, namely, that marketed as Glucophage® (trademark of Bristol-Myers Squibb Company).

The pharmaceutical composition of the invention can be formulated as an oral unit dosage form, for example as a tablet, pill, pellet, capsule, powder, lozenge, granule, solution, suspension, emulsion, syrup, elixir, oral liquid preparation, edible food product, prebiotic, probiotic, beverage, food bar, smoothie, shake, or yogurt. It may further comprise one or more optional agents selected from sweetening agents, flavoring agents, coloring agents, preserving agents, time delay or delay disintegration materials, standard oral vehicles, suitable carriers, excipients, or diluents.

It may optionally include one or more fillers or excipients such as lactose, sugar, corn starch, modified corn starch, mannitol, sorbitol, inorganic salts such as calcium carbonate and/or cellulose derivatives such as wood cellulose and microcrystalline cellulose.

One or more binders may be present in addition to or in lieu of the fillers. Examples of such binders which are suitable for use herein include polyvinylpyrrolidone, lactose, starches such as corn starch, modified corn starch, sugars, gum acacia and the like as well as a wax binder in finely powdered form such as carnauba wax, paraffin, spermaceti, polyethylenes or microcrystalline wax.

Where the pharmaceutical composition is to be in the form of a tablet, it will include one or more tableting lubricants such as magnesium stearate, stearic acid, palmitic acid, calcium stearate, talc, carnauba wax and the like. Other conventional ingredients which may optionally be present include preservatives, stabilizers, anti-adherents or silica flow conditioners or glidants, such as Syloid brand silicon dioxide as well as FD&C colors.

Tablets may also include a coating layer. The coating layer which is applied over the outer solid phase containing particles of inner solid phase embedded therein may comprise any conventional coating formulations and will include one or more film-formers or binders, such as a hydrophilic polymer like hydroxypropylmethylcellulose, and/or a hydrophobic polymer like methacrylic acid esters neutral polymer, ethyl cellulose, cellulose acetate, polyvinyl alcohol-maleic anhydride copolymers, β-pinene polymers, glyceryl esters of wood resins and the like and one or more plasticizers, such as triethyl citrate, diethyl phthalate, propylene glycol, glycerin, butyl phthalate, castor oil and the like. Both core tablets as well as coating formulations may contain aluminum lakes to provide color.

The film formers are applied from a solvent system containing one or more solvents including water, alcohols like methyl alcohol, ethyl alcohol or isopropyl alcohol, ketones like acetone, or ethylmethyl ketone, chlorinated hydrocarbons like methylene chloride, dichloroethane, and 1,1,1-trichloroethane.

Where a color is employed, the color will be applied together with the film former, plasticizer and solvent compositions.

The finished dosage form can be either a compressed tablet or a hard gelatin capsule, preferably a tablet. The tablet may be optionally film coated. The total amount of drug per dosage unit would be such as to offer a dosage form of convenient size for patients.

The pharmaceutical composition of the invention is preferably administered by oral administration. In some embodiments, the pharmaceutical composition may be administered by intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration.

The composition of the invention can have an acute effect that can be seen in less than one month. Additionally or alternatively, the composition can have a longterm effect, and thus various embodiments comprise administration of the composition to the individual (e.g., orally) for a time period of at least one month; preferably at least two months, more preferably at least three, four, five or six months; most preferably for at least one year. During the time period, the composition can be administered to the individual at least one day per week; preferably at least two days per week, more preferably at least three, four, five or six days per week; most preferably seven days per week. The composition can be administered in a single dose per day or in multiple separate doses per day.

As used herein, an “AMPK activator” refers to a compound that either increases the phosphorylation of downstream substrates of (phosphorylated or not) AMPK, and/or that increases the phosphorylation or enzymatic activity of AMPK.

As used herein, a “direct AMPK activator” refers to a compound that activates AMPK via direct interaction with the AMPK trimeric complex structure through at least binding one of its subunits.

As used herein, a condition, disorder, or disease “responsive to AMPK activation” refers to one in which the symptoms would be alleviated, or the course of which would be beneficially modified, through activation of AMPK, including without limitation, cancer, NAFLD, a metabolic disorder, diabetes, dyslipidemia, hypertension, being overweight, and obesity.

As used herein, the term “diabetes” includes insulin-dependent diabetes mellitus (i.e. IDDM, also known as type 1 diabetes) non-insulin-dependent diabetes mellitus (i.e. NIDDM, also known as type 2 diabetes), and prediabetes. Type 1 diabetes is the result of an absolute deficiency of insulin, the hormone which regulates glucose utilization. Type 2 diabetes often occurs in the face of normal, or even elevated levels of insulin and appears to be the result of the inability of tissues to respond appropriately to insulin. This is termed “insulin resistance”. Most type 2 diabetic patients are also overweight or obese. One of the criteria for diagnosing diabetes is the fasting plasma glucose level. A diabetic subject has a fasting plasma glucose level of greater than or equal to 126 mg/dl. A prediabetic subject is someone suffering from prediabetes. A prediabetic subject is a subject with impaired fasting glucose (a fasting plasma glucose level of greater than or equal to 100 mg/dl and less than 126 mg/dl); or impaired glucose tolerance (a 2-hour plasma glucose level of ≥140 mg/dl and <200 mg/dl); or insulin resistance, resulting in an increased risk of developing diabetes. Prevention of type 2 diabetes includes treatment of prediabetes.

As used herein, the term “dyslipidemia” encompasses abnormal levels of any lipid fractions as well as specific lipoprotein abnormalities. For example, it refers to elevation of plasma cholesterol and/or elevation of triglycerides and/or elevation of free fatty acids and/or low high-density lipoprotein (HDL) level and/or high low-density lipoprotein (LDL) level and/or high very low-density lipoprotein (VLDL) level. Dyslipidemia may for example contribute to the development of atherosclerosis and ultimately symptomatic vascular disease including coronary heart disease. Dyslipidemia may or may not be associated with diabetes.

As used herein, the term “metabolic disorder” encompasses any abnormal chemical and enzymatic reactions disrupting normal metabolism due to environmental and genetic factors (environmental factors include physical activity, nutrition), leading to excessive levels or deficiency of certain substances and dysfunction of energy homeostasis. Non-limiting examples of metabolic disorders include diabetes, NAFLD, dyslipidemia, hypertension, being overweight, obesity, and any combination thereof.

As used herein, “AMPK-related diseases” includes pathologic or pathogenomic conditions in which the activation of AMPK provides a salutary effect. Examples of such diseases or conditions include obesity, diabetes, metabolic syndrome, acute inflammatory lung injury, heart disease, reperfusion ischemia, cancer, aging, retinal degeneration, cardiac hypertrophy, non-alcoholic fatty liver disease, hypertension, albuminuria, sporadic Alzheimer's disease, muscular dystrophy, and osteoarthritis.

“Prevention” or “preventing” includes reduction of risk and/or severity of a condition, disorder, or disease.

The terms “treatment,” “treating,”, “treat”, “attenuate” and “alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder, and include treatment of patients at risk of contracting a disease or suspected to have contracted a disease, as well as patients who are ill or have been diagnosed as suffering from a disease or medical condition. The term does not necessarily imply that a subject is treated until total recovery. These terms also refer to the maintenance and/or promotion of health in a subject not suffering from a disease but who may be susceptible to the development of an unhealthy condition. These terms are also intended to include the potentiation or otherwise enhancement of one or more primary prophylactic or therapeutic measure. The terms “treatment,” “treat,” “attenuate” and “alleviate” are further intended to include the dietary management of a disease or condition or the dietary management for prophylaxis or prevention a disease or condition. A treatment can be patient- or doctor-related.

Obesity, which is an excess of body fat relative to lean body mass, is a chronic disease that is highly prevalent in modern society. It is associated not only with a social stigma, but also with decreased life span and numerous medical problems, including adverse psychological development, coronary artery disease, hypertension, stroke, diabetes, hyperlipidemia, and some cancers, (see, e.g., Nishina, et al., Metab. 43:554-558, 1994; Grundy and Barnett, Dis. Mon. 36:641-731, 1990; Rissanen, et al., British Medical Journal, 301:835-837, 1990).

Obesity related disorders are diseases or conditions where excessive body weight or high “body mass index (BMI)” has been implicated in the progression or suppression of the disease or condition. Representative examples of obesity related disorders include, without limitation diabetes, diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia. See, Harrison's Principles of Internal Medicine, 13th Ed., McGraw Hill Companies Inc., New York (1994). Examples, without limitation, of inflammatory conditions include diseases of the digestive organs (such as ulcerative colitis, Crohn's disease, pancreatitis, gastritis, benign tumor of the digestive organs, digestive polyps, hereditary polyposis syndrome, colon cancer, rectal cancer, stomach cancer and ulcerous diseases of the digestive organs), stenocardia, myocardial infarction, sequelae of stenocardia or myocardial infarction, senile dementia, cerebrovascular dementia, immunological diseases and cancer in general.

The term “subject” or “individual” means any animal, including a human, that could benefit from one or more of the compounds, compositions or methods disclosed herein. Generally, the subject is a human or an avian, bovine, canine, equine, feline, hircine, lupine, murine, ovine or porcine animal. The subject can be a “companion animal”, which is any domesticated animal, and includes, without limitation, cats, dogs, rabbits, guinea pigs, ferrets, hamsters, mice, gerbils, horses, cows, goats, sheep, donkeys, pigs, and the like. Preferably, the subject is a human or a companion animal such as a dog or cat. The subject can be elderly or an older adult.

The term “elderly” in the context of a human means an age from birth of at least 60 years, preferably above 63 years, more preferably above 65 years, and most preferably above 70 years. The term “older adult” in the context of a human means an age from birth of at least 45 years, preferably above 50 years, more preferably above 55 years, and includes elderly subjects. For other animals, an “older adult” has exceeded 50% of the average lifespan for its particular species and/or breed within a species. An animal is considered “elderly” if it has surpassed 66% of the average expected lifespan, preferably if it has surpassed the 75% of the average expected lifespan, more preferably if it has surpassed 80% of the average expected lifespan. An elderly cat or dog has an age from birth of at least about 7 years.

As used herein, an “effective dose” or “effective amount” is an amount that prevents a deficiency, treats a disorder, condition, or disease in a subject or, more generally, reduces symptoms, manages progression of the diseases or provides a nutritional, physiological, or medical benefit to the subject. The relative terms “improved,” “increased,” “enhanced” and the like refer to the effects of the composition disclosed herein relative to a composition lacking one or more ingredients and/or having a different amount of one or more ingredients, but otherwise identical.

As used herein, the singular forms “a,” “an” and “the” include plural referents unless the context clearly dictates otherwise. Thus, for example, reference to “a component” or “the component” includes two or more components.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of recombinant DNA technology include Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology include Goodman and Gilman's The Pharmacological Basis of Therapeutics, 10th Ed., McGraw Hill Companies Inc., New York (2001). Standard medical terminology used herein has the meaning defined in Stedman's Medical Dictionary, 27th Edition, with veterinary medicine insert.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or compounds, but may also include additional features or compounds. The term “and/or” used in the context of “X and/or Y” should be interpreted as “X,” or “Y,” or “X and Y.” Where used herein, the terms “example” and “such as,” particularly when followed by a listing of terms, are merely exemplary and illustrative and should not be deemed to be exclusive or comprehensive.

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the claims. Numerous specific details are set forth in the description in order to provide a thorough understanding of the present invention. The present invention may be practiced without some or all of these specific details.

In other instances, well known methods and protocols have not been described in detail, in order not to unnecessarily obscure the present invention.

### BRIEF DESCRIPTION OF FIGURES

FIG. 1. AMP and Lusianthridin activation of bacterially-expressed AMPKα2β1γ1. Lusianthridin (7-Methoxy-9,10-dihydrophenanthrene-2,5-diol, CAS number 87530-30-1) in combination with AMP result in additive activation of AMPK. AMP binding through the AMPKγ subunit causes a dose-dependent increase in AMPK activity and addition of various concentrations of Lusianthridin cause an additive activation of AMPK, without affecting the EC50 of AMP activation.

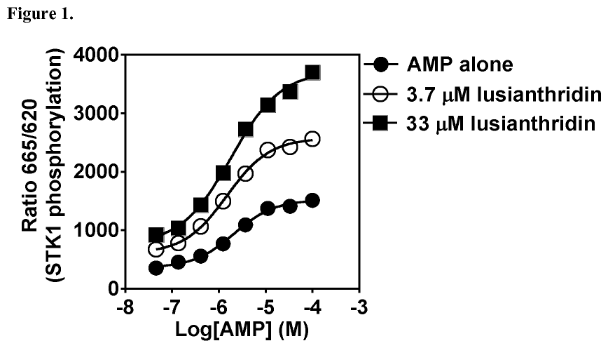

FIG. 2. Low concentrations of Lusianthridin alone and Metformin alone, cause an inhibition of lipogenesis. There is a further increase in lipogenesis when these two activators are used in combination.

### EXAMPLES

### Example 1

Lusianthridin Causes an Additive Effect on AMP Activation of Activate Bacterially-Expressed AMPKα2β1γ1 Complex.

The AMPK heterotrimers were expressed in bacteria and purified through the His-α subunit by nickel purification, further purified through gel filtration and finally phosphorylated by incubation with CaMKKβ. This phosphorylated AMPK was incubated with varying concentrations of AMP for 30 mins using substrate and reagents from the HTRF-KinEASE Cisbio assay kit (STK S1 Kit). Phosphorylation of the substrate was measured by incubating with donor and acceptor antibodies for 2 h at room temperature as per the manufacturer's protocol (and Coulerie et al., (2016), see below) and phosphorylated peptide detected by performing HTRF. The 665 nm/620 nm ratio was determined and displayed in the graph. In the presence of fixed concentrations of Lusianthridin, there was an additive activation of AMPK by AMP.

Reference 1: Standardized LC×LC-ELSD Fractionation Procedure for the Identification of Minor Bioactives via the Enzymatic Screening of Natural Extracts. Coulerie P, Ratinaud Y, Moco S, Merminod L, Naranjo Pinta M, Boccard J, Bultot L, Deak M, Sakamoto K, Queiroz E F, Wolfender J L, Barron D. J Nat Prod. 2016 Nov. 23; 79(11):2856-2864. Epub 2016 Oct. 28.

### Example 2

Lusianthridin and Metformin have an Additive Effect on Inhibition of Lipogenesis in Primary Hepatocytes.

Hepatocyte isolation: The liver was first perfused with 50 ml perfusion buffer (Krebs-Hepes buffer with 0.5 μM EDTA), followed with 50 ml collagenase A buffer (Krebs-Hepes buffer with 5 mM CaCl₂) and 0.5 mg/ml collagenase). After passage through a 100 μm mesh, the cell solution was washed several times with cold media and finally the cell culture pellet was resuspended in culture medium (medium 199 (M199)+GlutaMAX, 100 U/ml penicillin G, and 100 μg/ml streptomycin, 0.1% (wt/vol) BSA, 10% FCS, 10 nM insulin, 200 nM triiodothyronine and 500 nM dexamethasone). Hepatocytes were left to attach (3-4 h) and cultured overnight in M199 supplemented with antibiotics and 100 nM dexamethasone. Cells were used for experiments the following morning.

For lipogenesis measurements in primary hepatocytes, cells were seeded at 600 K cells per well in a 6-well plate overnight. Media was replaced with fresh M199 media alone for 2 hours prior to incubation with varying concentrations of Lusianthridin or compound 1 for 1 h at 37 C, in the presence of [1-¹⁴C]-acetate. The incorporation of [¹⁴C] into fatty acids was determined in the lower organic layer after separation from the aqueous phase. The results are displayed as the disintegrations per min (DPM) per μg of protein.

Lipogenesis is controlled by the AMPK substrate ACC, and phosphorylation and inhibition of ACC by AMPK, leads to a decrease in lipogenesis. Lipogenesis was measured in primary hepatocytes by determining the incorporation of ¹⁴C-labelled acetate into fatty acids.

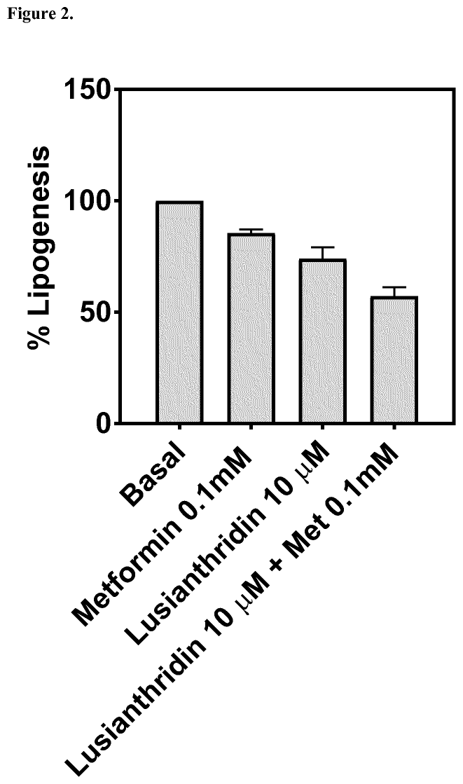

Lipogenesis was monitored in the presence or absence of a low/non-saturating concentration of Lusianthridin or metformin for 1 h at 37 C. Furthermore, cells were incubated in the presence of both of these compounds. The results shown in FIG. 2, displayed as the % lipogenesis (compared to control untreated cells), that Lusianthridin and metformin are able to inhibit lipogenesis. When combined, Lusianthridin and metformin cause a further inhibition of lipogenesis. These data suggest that when low concentrations of AMPK activators are combined, there is a greater inhibition of lipogenesis compared to the effect of these activators alone.

1. A medicament comprising
(i) Metformin; and
(ii) a compound having the general formula (I) 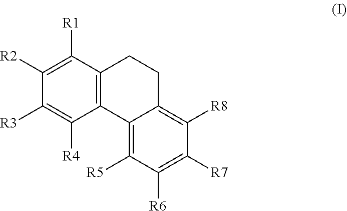
wherein, in compound (ii), R1, R2, R3, R4, R5, R6, R7, and R8 are each independently H; OH; OMe; O-glycoside; a halogen; an aldehyde; a carboxylic acid; a primary, secondary, or tertiary amine; a primary or secondary amide; a cyano; a nitro; a sulfonate; and a sulfate.

2. The medicament according to claim 1, wherein R1, R2, R3, R4, R7, and R8 are each independently H; OH; OMe; O-glycoside; a halogen; an aldehyde; a carboxylic acid; a primary, secondary, or tertiary amine; a primary or secondary amide; a cyano; a nitro; a sulfonate; a sulfate; R5 is H; OMe; O-glycoside; a halogen; an aldehyde; a carboxylic acid; a primary, secondary, or tertiary amine; a primary or secondary amide; a cyano; a nitro; a sulfonate; a sulfate; O-glycoside; a halogen; an aldehyde; a carboxylic acid; a primary, secondary, or tertiary amine; a primary or secondary amide; a cyano; a nitro; a sulfonate; and a sulfate.

3. The medicament according to claim 1, wherein R1, R2, R3, R4, R7, and R8 are each independently H; OH; OMe; O-glycoside; a halogen; an aldehyde; a carboxylic acid; a primary, secondary, or tertiary amine; a primary or secondary amide; a cyano; a nitro; a sulfonate; a sulfate; R5 is H; OMe; O-glycoside; a halogen; an aldehyde; a carboxylic acid; a primary, secondary, or tertiary amine; a primary or secondary amide; a cyano; a nitro; a sulfonate; a sulfate; R6 is H; OH; O-glycoside; a halogen; an aldehyde; a carboxylic acid; a primary, secondary, or tertiary amine; a primary or secondary amide; a cyano; a nitro; a sulfonate; and a sulfate in compound (ii).

4. The medicament according to claim 1, wherein R1, R3, and R8 are each independently H; OH; OMe; O-glycoside; a halogen; an aldehyde; a carboxylic acid; R2 is OH; OMe; O-glycoside; a halogen; an aldehyde; a carboxylic acid; R4 is H; OH; O-glycoside; a halogen; an aldehyde; a carboxylic acid; R5 is H; OMe; O-glycoside; a halogen; an aldehyde; a carboxylic acid; R6 is H; OH; O-glycoside; a halogen; an aldehyde; a carboxylic acid; and R7 is H; OH; O-glycoside; a halogen; an aldehyde; and a carboxylic acid in compound (ii).

5. The medicament according to claim 1, wherein R1, R3, and R8 are each independently H; OH; OMe; R2 is OH; OMe; R4 is H; OH; R5 is H; OMe; R6 is H; OH; and R7 is H; and OH in compound (ii).

6. The medicament according to claim 1, wherein R1, R3, R6 and R8 are each independently H; R2 is OH; OMe; R4 is OH; R5 is H; OMe; and R7 is H; and OH in compound (ii).

7. The medicament according to claim 1, wherein compound (ii) is Lusianthridin (7-Methoxy-9,10-dihydrophenanthrene-2,5-diol, CAS number 87530-30-1) and has the formula

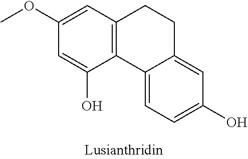 [SMILES: COc1cc(O)c2c(c1)CCc1cc(O)ccc1-2]

8. The medicament according to claim 1, wherein Metformin or a salt thereof, and a compound of general formula (I), or a salt thereof are combined in a single preparation.

9. The medicament according to claim 1, wherein Metformin or a salt thereof, and a compound of general formula (I), or a salt thereof are separately formulated into compositions and used in combination.

10. A pharmaceutical composition comprising a medicament comprising
(i) Metformin; and
(ii) a compound having the general formula (I) 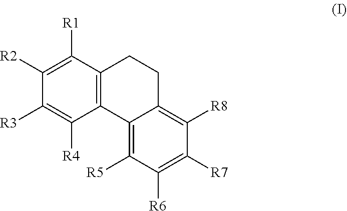
wherein, in compound (ii), R1, R2, R3, R4, R5, R6, R7, and R8 are each independently H; OH; OMe; O-glycoside; a halogen; an aldehyde; a carboxylic acid; a primary, secondary, or tertiary amine; a primary or secondary amide; a cyano; a nitro; a sulfonate; and a sulfate, in a pharmaceutically acceptable form.

11. The pharmaceutical composition of claim 10, for use in the prevention or treatment of a metabolic disease or a cardiovascular disease.

12. The pharmaceutical composition according to claim 11, wherein the metabolic disease is diabetes.

13. The pharmaceutical composition according to claim 12, wherein the prevention or treatment of diabetes is by increasing the inhibition of lipogenesis.

14. The pharmaceutical composition according to claim 13, wherein the prevention or treatment of diabetes is by increasing the inhibition of lipogenesis in liver cells and improving insulin sensitivity by activation of AMP-activated protein kinase (AMPK).

15. The pharmaceutical composition according to claim 9, wherein metformin is used at a sub-pharmalogical dose for the subject.

16. The pharmaceutical composition according to claim 15, wherein the sub-pharmacological dose is not more than about 50% of the standard pharmacological dose for the subject.

17. The pharmaceutical composition according to claim 14, wherein the subpharmacological dose of metformin reduces the incidence of side effects associated with metformin use, wherein the side effects are lactic acidosis, and/or gastro-intestinal side effects, particularly diarrhea, nausea, and vomiting.

18. A method of preventing or treating a metabolic disease or a cardiovascular disease comprising the administration of a medicament comprising
(i) Metformin; and
(ii) a compound having the general formula (I) 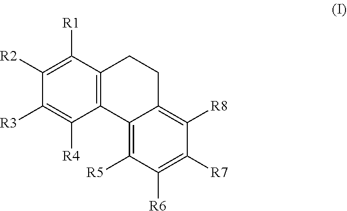
wherein, in compound (ii), R1, R2, R3, R4, R5, R6, R7, and R8 are each independently H; OH; OMe; O-glycoside; a halogen; an aldehyde; a carboxylic acid; a primary, secondary, or tertiary amine; a primary or secondary amide; a cyano; a nitro; a sulfonate; and a sulfate to a subject in need thereof.

19. The method according to claim 18, wherein the disease is diabetes.
